(19) United States
(12) Patent Application Publication
(10) Pub. No.: US 2017/0197082 A1
(43) Pub. Date: Jul. 13, 2017

(54) DEVICE, SYSTEM AND METHOD FOR NERVE STIMULATION

(71) Applicants: Dexing PANG, New York, NY (US); Qiongzhen LONG, Singapore (SG)

(72) Inventors: Dexing PANG, New York, NY (US); Qiongzhen LONG, Singapore (SG)

(21) Appl. No.: 15/325,023

(22) PCT Filed: Jul. 9, 2015
(86) PCT No.: PCT/SG2015/050209

§ 371 Date: Jan. 9, 2017

(30) Foreign Application Priority Data
Jul. 9, 2014 (CN) 201410323604
Jul. 9, 2014 (CN) 201420375909

(51) Int. Cl. A61N 1/36; A61N 1/08; A61N 1/372; A61N 1/05; A61N 1/378

(52) U.S. Cl. CPC A61N 1/36071; A61N 1/0551; A61N 1/3782; A61N 1/37223; A61N 1/08; A61N 1/37205

(57) ABSTRACT

The present invention is a nerve stimulation system for treating pain in a patient, the system comprising an implantable device (100) having: a housing (18) having a power source; and at least one stimulation electrode (17) arranged on the housing (18) and in electrical communication with the power source, the stimulation electrode (17) adapted to transmit an electrical signal for stimulating at least one nerve cell of the patient, and wherein the power in the power source is wirelessly generated. The fact that the device (100) at least one stimulation electrode (17) arranged on the housing (18) advantageously provides a compact and miniature device for simple, minimally invasive implantation into a patient to treat pain. The size of said device (100) allows it to work with commomly used injectors such as a standard medical syringe and a stainless steel needle. Further, the device (100) can be powered wirelessly, which allows said device (100) to be implanted for a long period of time.

### FIELD OF INVENTION

The present invention relates to devices, systems and methods for nerve stimulation.

### BACKGROUND OF INVENTION

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

There are many diseases and/or disorders related to injuries of the nervous system, including injuries to the central, peripheral and autonomous nervous system, which can induce sensory disturbances, movement disorders and conscious disturbances. Patients with such diseases and/or disorders may experience varying degrees of pain. Pain, for example chronic pain which is commonly understood as pain lasting longer than three to six months, affects a person's quality of life, for example causing sleep disturbances and impairing the ability to work.

Several treatments for pain are currently available. Traditionally, medication such as analgesics has been used to treat or reduce pain, where such drugs typically act in various ways on the central and peripheral nervous system. Certain medication such as anti-inflammatory drugs and steroids act directly on the nociceptive injury to alleviate pain. An example of a medication for treating pain experienced by cancer patients is disclosed in Chinese patent application number 201310537668. Although pain treatment by medication is commonly used, such methods have long pathological response time, short duration of action and may have undesirable side effects.

Chinese patent no. 203564311U discloses a method of pain treatment using acupuncture. This method expands the range of pain treatments available to patients. However, this method is difficult to operate and has limited treatment ranges. Due to the nature of the procedure disclosed in this patent, the electric field for the treatment is only efficient on the skin surface. Chinese patent no. 203355134U discloses a post-operative pain treatment instrument which achieves pain treatment by working on a patient's skin. This method is simple and can be applied to a wide range of diseases. However its therapeutic effect is poor and needs improvement because its working electrode is difficult to locate and it only works on skin. Chinese patent no. 302012071S discloses an external RF (radio frequency) device for pain treatment. This device utilizes high-frequency electromagnetic waves which are capable of penetrating a patient's skin surface and is mainly used to treat nerve system pains.

Besides medication and acupuncture, electrical nerve stimulation is a procedure that uses an electrical current to treat pain. Such nerve stimulation has been a well-accepted clinical treatment method for patients. U.S. Pat. No. 6,895,280 B2 discloses a spinal cord stimulator (SCS). This stimulator comprises an implantable pulse generator with attachable working electrodes that extend to the relevant spinal nerves when the generator is preferably implanted in the abdomen or just above the buttocks. While this device is effective for a variety of nervous system disorders, such as reflex nerve disorders (RSD), said device has a complicated design and structure, is difficult to implant, has high manufacture costs, and also includes an internal power supply which requires charging/replacement once the power in the device has been used up charging/replacement of the power supply requires the surgical removal of the device.

Therefore the object of the present invention is to provide for an implantable nerve stimulation device for stimulating nerve tissue and/or cells in a patient for the treatment of pain.

### SUMMARY OF INVENTION

Throughout the specification, unless the context requires otherwise, the word “comprise” or variations such as “comprises” or “comprising”, are to be construed as inclusive and not exhaustive.

Furthermore, throughout the specification, unless the context requires otherwise, the word “include” or variations such as “includes” or “including”, are to be construed as inclusive and not exhaustive.

In a first embodiment, the present invention is a nerve stimulation system device for treating pain in a patient, the system comprising an implantable device having: a housing having a power source; and at least one stimulation electrode arranged on the housing and in electrical communication with the power source, the stimulation electrode adapted to transmit an electrical signal for stimulating at least one nerve cell of the patient, and wherein the power in the power source is wirelessly generated. The fact that the device has at least one stimulation electrode arranged on the housing advantageously provides a compact and miniature device for simple, minimally invasive implantation into a patient to treat pain. The size of said device allows it to work with commonly used injectors such as a standard medical syringe and a stainless steel needle. Further, the device can be powered wirelessly, which allows said device to be implanted for a long period of time.

Preferably, the device further comprises at least one reference electrode arranged on the housing.

Preferably, the housing further has a first end and a second end, and wherein the stimulation electrode is arranged at the first end and the reference electrode is arranged at the second end.

It is preferred that the nerve stimulation system further comprises at least one transceiver, wherein the device is configured to be in data communication with the transceiver, and the transceiver configured to provide instructions to the device to generate and transmit the electrical signal for stimulating at least one nerve cell in the patient. Preferably, the transceiver is configured to induce electrical power in the device.

Preferably, the device further includes a processor configured to communicate with the transceiver, and preferably, such communication is a wireless communication. Preferably the device is configured to wirelessly communicate with the transceiver via radio frequency (RF) and more preferably via radio frequency identification (RFID). The processor is also configured to generate the electrical signal. Preferably, the processor is an ASIC. Preferably, the processor is configured to control the impedance in the device and to optimize ground loop impedance.

Preferably, the device includes a temperature transducer configured to monitor the temperature of the device or optionally the temperature of the environment surrounding the device. It is preferred that the device includes an antenna.

Preferably, the device has more than one stimulation electrode. Preferably, each of the stimulation and reference electrodes has a diameter ranging from 1 μm to 200 μm in one dimension. Preferably each of the stimulation and reference electrodes has a diameter of 100 μm.

Preferably, the device is configured to select the stimulation electrodes for transmission of the electrical signal and it is preferred that the distance between each stimulation electrode ranges from 1 μm to 500 μm. Preferably the distance between each stimulation electrode is 200 μm.

Preferably, the housing includes a biocompatible coating, where said coating includes, but not limited to parylene or polyether ether ketone (PEEK).

Preferably, the device is implantable in a patient via injection. The device is implantable up to 5 cm from the surface of the patient's skin.

Preferably the transceiver is configured to provide instructions to the device for the selection of the stimulation electrodes for transmission of the electrical signal. It is preferred that the transceiver is configured to receive data from the device.

In a second embodiment, the present invention provides a nerve stimulation implantable device comprising a housing having a power source; and at least one stimulation electrode arranged on the housing and in electrical communication with the power source, the stimulation electrode adapted to transmit an electrical signal for stimulating at least one nerve cell of the patient, wherein the power in the power source is wirelessly generated.

Preferably, the device further comprises at least one reference electrode arranged on the housing.

Preferably, the housing has a first end and a second end, and wherein the stimulation electrode is arranged at the first end and the reference electrode is arranged at the second end.

Preferably the device further comprising a processor configured to generate the electrical signal. Preferably the processor is configured to control the impedance in the device and to optimize ground loop impedance.

Preferably the device includes a temperature transducer configured to monitor the temperature of the device, or optionally the temperature of the environment surrounding the device.

Preferably, the device has more than one stimulation electrode.

Preferably, each of the stimulation and reference electrodes has a diameter ranging from 1 μm to 200 μm in one dimension. Preferably each of the stimulation and reference electrodes has a diameter of 100 μm.

Preferably the device is configured to select the stimulation electrodes for transmission of the electrical signal and it is preferred that the distance between each stimulation electrode ranges between 1 μm to 500 μm. Preferably the distance between each stimulation electrode is 200 μm.

In a third embodiment, the present invention provides a method of treating pain in a patient, the method comprising the steps of: implanting at least one implantable device at an implantation site of the patient, the device having: a housing having a power source and at least one stimulation electrode arranged on the housing and in electrical communication with the power source, the stimulation electrode adapted to transmit an electrical signal for stimulating at least one nerve cell of the patient; bringing at least one transceiver to the implantation site, wherein the device is configured to be in data communication with the transceiver; and instructing the device via the transceiver to generate and transmit via the stimulation electrode, an electrical signal for stimulating at least one nerve cell in the patient, wherein the power in the power source is wirelessly generated.

### BRIEF DESCRIPTION OF FIGURES/DRAWINGS

The present invention will now be described, by way of example only, with reference to the accompanying drawings, in which:

FIG. 1 provides a schematic view of a first embodiment of an implantable nerve stimulation device according to the present invention.

FIG. 2 provides a flow chart illustrating the generation and transmission of an electrical signal within an embodiment of an implantable nerve stimulation device according to FIG. 1.

FIG. 3 provides a circuit block diagram of the interface and stimulation circuitry of a second embodiment of an implantable nerve stimulation device according to the present invention.

FIG. 4 provides a representative diagram illustrating the second embodiment of an implantable nerve stimulation device according to FIG. 3 with an external transceiver.

Other arrangements of the invention are possible and, consequently, the accompanying drawings are not to be understood as superseding the generality of the preceding description of the invention.

### DETAILED DESCRIPTION OF INVENTION

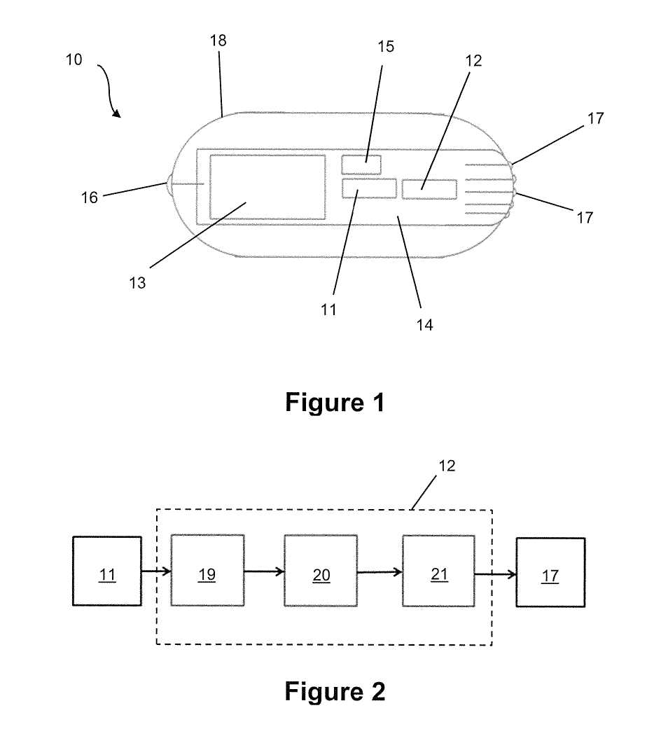

Referring now to the drawings which are for the purposes of illustrating various aspects of the invention and not for purposes of limiting the same. FIG. 1 provides a schematic representation of an embodiment of an implantable nerve stimulation device according to the present invention. The term “implantable” is taken herein to include placing the device within the body of a patient without any exposed portions, such that when implanted, the device is substantially surrounded by the cells of the tissue in which it is intended to be placed.

The term “patient” is used throughout the specification to describe an animal, preferably a human, to whom treatment is provided. For treatment of those conditions which are specific for a specific animal such as a human patient, the term patient refers to that specific animal.

The term “treatment” is understood to include anything done or provided for alleviating or preventing the effects or symptoms of a disease or disorder, whether it is done or provided by way of cure or not. A reduction in any particular symptoms of the disease or disorder the present invention is intended for, resulting from practising the present invention, is considered alleviation of the symptom.

“Nerve tissue” used herein refers to a composition of neurons, or nerve cells which are commonly known to receive and/or transmit impulses, and it also includes neuroglia which aids to propagate nerve impulses and provides nutrients to the nerve cells. Nerve tissue can form part of the central nervous system and the peripheral nervous system. Nerve tissue used herein can also be taken to refer to a single nerve cell, whole or part thereof.

The term “stimulate” or variations such as “stimulating” used herein in relation to cells and tissue, refers to an excitation or desensitization of that cell and/or tissue, or provision of a current which bypasses that cell and/or tissue to reach other cells and/or tissues.

“Power source” used herein refers to at least one electrical component for storing and/or providing electrical energy. The power source may comprise electrical modules or elements to receive electrical energy via a remote wireless means. Such elements include (but are not limited to) capacitors.

Referring to a first embodiment of the present invention as shown in FIG. 1, an implantable nerve stimulation device 10 includes a baseband module 11, a processor 12, an antenna 13, and additional circuit components 15. Additional circuit components 15 are components required to stabilize the circuit and include but are not limited to capacitors, resistors and inductors. Additional circuit components 15 may be in a surface mountable package. The processor 12, antenna 13 and additional circuit components 15 are preferably arranged, mounted and/or soldered on a printed circuit board 14. The various components 11, 12, 13, 14, and 15 may be enclosed within a housing 18.

The baseband module 11 is operable to receive and modulate any signals received from the antenna 13. Baseband module 11 may be in the form of an integrated circuit chip, and comprises analog and digital components.

The device 10 also includes reference electrodes 16 and stimulation electrodes 17 which have a portion arranged on the external surface of a housing 18. Part of the reference electrodes 16 and the stimulation electrodes 17 extends into the housing 18 to electrically connect, with the baseband module 11 and the processor 12, various internal components and a ground electrode (not shown) of the device 10. The electrodes 16, 17 are preferably needle-shaped with a cross-sectional diameter of 100 μm. It will be appreciated that the size of each electrode 16, 17 will depend on the type of tissue and/or cell being treated and the type of problems associated with said tissue and/or cell. Accordingly, the cross-sectional diameter of each electrode 16, 17 can range from a few μm to a few hundred μm, for example from 1 μm to 200 μm. It is preferable that the cross-sectional diameter of each electrode 16, 17 is 100 μm. It will also be appreciated that the size of each electrode 16, 17 can be based on dimensions other than the cross-sectional diameter of the electrode, for example the length, width or radius of the electrode, which in turn can depend on the shape of the electrode. Accordingly, the terms “dimension” or “dimensions” used throughout the specification includes but is not limited to cross-sectional diameters.

While FIG. 1 illustrates a single reference electrode 16 and an array of stimulation electrodes 17, it would be appreciated that a single stimulation electrode 17 may be arranged on the surface of the housing 18 and it would also be appreciated that an array of reference electrodes 16 may be used instead of a single reference electrode. An array of stimulation electrodes 17 is preferred to increase surface area for a more intensive electrical signal (which can be electro-magnetic in nature in an alternating current circuit) that will induce a stronger electrical stimulation and/or magnetic field simulation of the nerve tissue. When in an array, the distance between each electrode 17 can range from 1 μm to 500 μm and is preferably 200 μm. This distance of 200 μm is preferable because at least one electrode 17 will be best positioned to provide an optimal electrical signal for nerve cell and/or tissue stimulation. However it will be appreciated that the distance between the electrodes 17 may vary depending on the application, for example, the size of the tissue and/or cell being treated and the physical size of the device 10. The exposed portion of the stimulation electrode 17 on the housing 18 is preferably tapered, with the tapered point (i.e. the sharpest point) furthest away from the housing 18 so as to concentrate the electrical signal prior transmission and to increase the stimulation field discharging intensity. Preferably, the reference electrode 16 and the stimulation electrode 17 are arranged at each end of the housing 18, i.e. the reference electrode 16 and stimulation electrode(s) 17 are at opposite ends of the housing 18. This arrangement forms a symmetrical distribution of the electro-magnetic field around the two end point of the device 10 and guarantees a stable and reliable current loop. However, it is contemplated that they can also be arranged anywhere on the housing, for example along a middle portion of the housing 18. The reference electrode 16 and stimulation electrode 17 may be made from suitable biocompatible materials which include, but are not limited to titanium and gold, or metals coated in titanium or gold, where such coating may be achieved for example by physical vapor deposition or other techniques.

The housing 18 is a single unitary structure molded from plastics, preferably medical grade plastics, and is coated with a suitable biocompatible protective material which includes but is not limited to parylene or polyether ether ketone (PEEK), or nano-molecular compositions of the same. Parylene is preferred because it is used by standard 0.18 μm complementary metal-oxide semiconductor (CMOS) tape-out process, and is biocompatible. Such biocompatible materials provide protection against moisture, water, acids and alkalis, which forms part of the environment when device 10 is implanted into the body of a patient. Depending on the application and fabrication techniques of device 10, housing 18 need not be a unitary single structure and may be made from other materials such as metals.

The processor 12 comprises a digital-to-analog/analog-to-digital convertor (DAC/ADC) module 19, a pulse generation module 20 and a switch 21. It would be appreciated that the processor 12 is or includes an application specific integrated circuit (ASIC). The application-specific integrated circuit (ASIC) is programmable using a hardware description language. The ASIC may include microprocessor(s), memory blocks necessary for implementing logic to selectively activate or deactivate the stimulation electrode(s) 17 via the switch 21. The ASIC is an application specified unit, and it includes analog circuitry for the signal processing in the front end, DAC/ADC module 19 to convert the analog signal to digital signal, and to interface with baseband module 11. The baseband module 11, processor 12 and the stimulation electrodes 17 form the electrical signal generation circuit of the device 10.

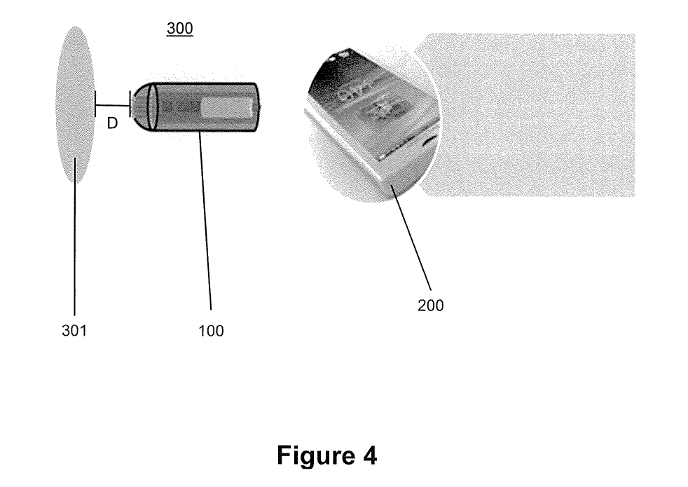

Upon receiving or detecting an electrical signal from an external transceiver 200, a potential difference or voltage is induced across the antenna 13 (FIG. 4). Part of the induced voltage is used to drive the baseband module 11, DAC/ADC 19, pulse generation module 20 and switch 21 (see FIG. 2). The pulse generation module 20 controls whether the electrical signal is to be transmitted in pulses and if so, controls the nature of the pulse, for example pulse length and frequency. A skilled person will be able to determine the pulse rate which can range from several hertz to several kilo hertz, and pulse pattern, depending on the location of the nerve cell and/or tissue, the type of disease treated and treatment rendered. The pulse generation module 20 further includes comparators, reference circuits, pre-drivers, level-shifter circuits and logic control modules. The switch 21 can be an analog or digital switch, such as, but not limited to a metaloxidesemiconductor field-effect transistor (MOSFET) or other electronic transistor and it is able to select which stimulation electrodes 17 are to transmit the electrical signal. The switch 21 can also control the components of the device 10 to alter the impedance of the internal circuitry. Depending on the application, other devices which include but are not limited to varicaps, may be used to alter the impedance of the internal circuitry. By selecting an optimal impedance for the ground loop, the efficiency of the stimulation electrodes 17 can be optimized.

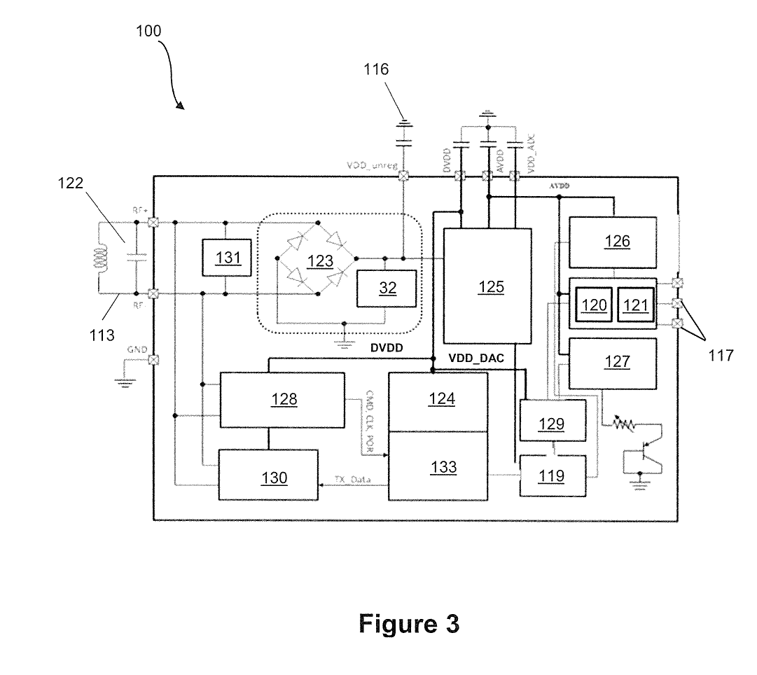

The implantable nerve stimulation device of the present invention may also be implemented in the form of an integrated chip as shown in FIG. 3 which provides another embodiment of the present invention. In particular, FIG. 3 provides a circuit block diagram of the interface and stimulation circuitry of device 100 as implemented in the form of an integrated chip. Antenna 113, which is preferably a radio frequency (RF) antenna, is operable to receive/send RF input/output (in the form of data packets) from/to an external transceiver 200; a rectifier module 123 operable to rectify the received RF input; a power management module 125 operable to receive the rectified RF input, a portion of the rectified RF input being used for powering module 125 . . . Upon power up, the power management module 125 is further operable to:
a. provide a first drive voltage AVDD for driving voltage generator 126, a temperature transducer 127, a pulse generation module 120, a switch 121 which selects which stimulation electrode 117 transmits the electrical signal for stimulating the nerve tissue, and stimulation electrodes 117;
b. provide a second drive voltage VDD_DAC for driving a multiplexer 129 and a DAC/ADC 119; and
c. provide a third drive voltage DVDD for driving other components such as a signal demodulator/clock extractor/power-on-reset 128; a load modulator 130; a storage unit 124 and digital baseband 133 etc.

An RF limiter 131 may be placed in parallel with the RF antenna 113 for RF circuit protection. Likewise the rectifier may comprise a voltage limiter 132 for circuit protection.

The device 100 does not contain a power supply and instead receives power wirelessly from the external transceiver 200 via electromagnetic induction, when the device 100 is in close proximity with the external transceiver 200. The antenna 113 receives a wireless signal from the external transceiver 200, which via induction generates an alternating current in the antenna and the alternating current is then used to provide power to the stimulation device 100. The antenna 113 also receives data from and communicates with the external transceiver 200 via RF, preferably RFID (radio frequency identification). The external transceiver 200 can, through the transmission of such data to antenna 113, adjust the electrical signal's output voltage, waveform and strength, and control which and how many of the stimulation electrodes 117 transmit the electrical signal. In operation, data is received in the form of data packets from the external transceiver 200 and power is induced by the external transceiver 200 via the antenna 113, and by a coupling module 122 (for example an L-C resonant circuit), rectifier module 123, power management module 125, and voltage generator 126, certain voltage is generated and transmitted as an electrical (or electro-magnetic) signal via stimulation electrodes 117 to the intended nerve tissue 301. A storage unit 124 stores the parameters and instructions received from the external transceiver 200, such as electrical voltage amplitude (which can range from 10 mV to 1000 mV), waveform, and pulse length of the electrical signal and stimulation electrodes index, which is information on the number of electrodes (i.e. one or more) to be used. The stimulation electrodes index is accessed by the switch 121 to determine which stimulation electrodes 117 transmit the electrical signal. The number and selection of electrodes depends on the feedback of the patient and can be determined by a skilled person or the device 100 having been provided with suitable pre-configured instructions. The digital baseband 133 works according to a pre-defined and pre-configured work flow, and the instructions from the external transceiver 200, and samples the signal from the temperature sensor 127 by means of the DAC/ADC 119. The temperature data is used to evaluate the working status of the device 100 and the tissue surrounding the device 100, to ensure that the surrounding tissue is not damaged by overheating of the device 100. If the device 100 is working normally and the temperature of device 100 is close to the temperature of the surrounding tissue, the pulse generation module 120 and the switch 121 are activated through the DAC/ADC 119 to generate an electrical signal for transmission through the stimulation electrodes 17. It would be appreciated that the switch 121 can be an analog or digital switch. The electrical signal stimulates the intended nerve tissue 301. The electrical signal can for example, stimulate the sympathetic ganglia, dorsal root ganglia, thalamus and cerebral cortex, through myelinated nerve fibers, non-myelinated nerve fibers, sympathetic fibers, spinal cord lateral hypothalamus. Such electrical stimulation includes but is not limited to the activation and de-activation cells and/or tissue, and the bypassing of damaged cells and/or tissue to downstream cells and/or tissue to complete signal transmission. Further, the electrical signal transmitted via the device 100, when implanted, can replace epidural stimulation and anesthetics which act on the spinal lateral hypothalamus and opioids which act on the thalamus. The device 100 can treat various diseases caused by nervous system injuries, such as reflex sympathetic dystrophy (RSD), and the electrical signal generated and transmitted by the device 100 can be applied to treat somatic, visceral and neuropathic pain. Based on a feedback, either feedback from the patient or a measurable signal transmitted to the external transceiver 200, upon activation of the electrical signal, an operator can adjust settings in the external transceiver 200 to select the optimum stimulation mode (e.g. which electrode 117 to be used, the pulse intensity, the treatment time and the pulse frequency) accordingly.

It is appreciated that pain is a subjective experience for different individuals and pain can be felt vastly different on different individuals. In a normal individual, it may be easy to observe tell-tale signs of pain—such as tearing, verbal communications and grasping of pained areas. However, in the case of the elderly, the impaired, the psychologically impaired, young children or individuals in severe injury warranting no verbal communications, it is important to watch out for signs of pain, or what nurses tend to term as ‘silent pain’. These signs could include restlessness, nervousness, sleep disturbances, respiration disruptions, blood pressure fluctuations, body positions and even minute facial expressions. Accordingly, pain intensity is unfortunately not easily represented by a scale. Historically a pain scale has four options—none, mild, moderate and severe. However, as time progressed, the more commonly used scale is the 10 point pain scale. The McGill pain questionnaire is also a well-known pain assessment for individuals where there is an abridged version comprising of 62 different aspects distributed in 15 sections and further divided into three classifications—sensory, affective and evaluation/temporary. Since then, minute adjustments to the questionnaire have been conducted to suit different needs of different settings. Other common scales for pain assessment can be found in the Pain Assessment Scales by the National Initiative on Pain Control, which in incorporated herein by reference. As there is no universally standard way to assess pain, a common method is to establish a standard operating procedure (SOP) which can be used when necessary. For example, the University of Kansas Hospital uses the following SOP:
1. Describe the pain with words
2. Intensity of the pain (numeric and word scale)
3. Location of the pain
4. Any aggravating/alleviating factors
5. Other factors contributing symptoms or side effects

An established SOP is important for accurate assessment of pain, and it should be tailored specifically for different purposes (i.e. trauma pain, chronic pain, etc.). It will accordingly be appreciated that a skilled person will be able assess the pain experienced by an individual based on the available pain scales and/or SOPs, and determine the settings in the external transceiver 200 to select the optimum stimulation mode (e.g. which electrode 117 to be used, the pulse intensity, the treatment time and the pulse frequency) for the treatment of that individual's pain via device 100. Alternatively, the device 100 or transceiver 200 may be suitably configured with an appropriate pain scale and/or SOP to determine the optimum stimulation mode for the treatment of an individual's pain via device 100. Preferably the pain scale used is the 10 point pain scale, where the device 100 may be configured to generate and transmit suitable electrical (or electro-magnetic) signals to treat the different types of pain experienced according to the different points on the 10 point pain scale. For example, an individual expressing a pain of 7 on the 10 point pain scale may be treated by setting device 100 to generate and transmit an electrical (or electro-magnetic) signal with a greater amplitude and intensity compared to an electrical signal generated and transmitted to treat an individual experiencing a pain of 2 on the 10 point pain scale.

The device 100 also sends data to the external transceiver 200, which is capable of scanning for data transmitted by the device 100. The data is multiplexed and converted to a digital data packet for feeding into the digital baseband 133, which converts the digital data packet into a transmission data packet to be sent to the external transceiver 200. The transmission data packet may be sent to a load modulator 130 for signal modulator before being sent to the external transceiver 200 via the antenna 113. The data which the device 100 sends to the external transceiver 200 can include information regarding the status and working condition of the device 100, the electrodes, pulse intensity, frequency utilised, duration of the treatment, temperature of the environment surrounding device 100 and also on the nature of the electrical signal generated and transmitted.

The device 100 is implantable into a patient via parenteral and/or enteral means, which include but not limited to intramuscular, intravenous, subcutaneous, oral or transdermal means. The device 100 is preferably implanted via direct injection into the target tissue 300 proximate or close to a nerve tissue 301. For example, the device 100 is implanted in the peripheral region of nociceptive injury, which is the site commonly targeted by anti-inflammation drugs and steroids. Alternatively, the device 100 may be directly implanted in the vicinity of the posterior root ganglion and sympathetic nerve, for better therapeutic effect. Depending on the application, the device 100 may be in direct contact with the nerve tissue 301. The device 100 may be used in post-operation pain treatment by targeting suitable nerve tissue sites, thereby providing better therapeutic effects comparing to many traditional methods, such as acupuncture, laser and infrared treatment. Implantation of the device 100 may be achieved by means commonly known in the art, for example if by way of injection, the device 100 may be implanted using a specially designed injector or standard medical injectors, e.g. syringe and a stainless steel needle with an inner diameter of 2 mm. It is contemplated that the site and depth of implantation of device 100 depends on its application which can be assessed by a skilled professional based on his medical knowledge and clinical experience, for example, the device 100 may be implanted 0-5 cm from the surface of the patient's skin near a nerve tissue. In cases where implantation may be complicated due to sensitivity of surrounding tissues, for example the implantation site is proximate the hypothalamus, the implantation of the device 100 may be assisted using x-ray. Due to its inert biocompatible coating and use of a wireless power supplied by external transceiver 200, the device 100 may be implanted in a patient from a short to a long term basis. A “short term” or “long term” basis depends on the disease and patient's condition and can be determined by a skilled person, whereby the device 100 can be implanted in the patient ranging from several days to several years. Depending on the application or the extent of the disease and/or disorder, or the treatment thereof, more than one device 100 may be implanted into a patient. Having more than one device 100 implanted in a patient, for example at a single tissue site or at multiple implantation sites, can achieve more reliable and better therapeutic effect. It is appreciated that a single external transceiver 200 may be able to communicate with more than one of the implanted devices 100. Alternatively, only one transceiver 200 can communicate with only one device 100, i.e. there being multiple external transceivers 200 if multiple devices 100 are implanted.

Electrical signal characteristics such as amplitude and waveform can depend on the distance D as shown in FIG. 4, which is the distance from the stimulation electrode 117 to the nerve tissue 301. If distance D is substantially large, the strength of the electrical signal may be increased to effect the intended stimulation of the nerve tissue 301. That is, the longer distance D, the larger the electrical signal. Depending on distance D, a skilled person will be able to ascertain the appropriate signal strength required for optimal treatment.

If treatment is completed or device 100 needs repair, device 100 may be extracted by a specially designed extractor suitable for removing said device 100, if the implantation depth is less than 2 cm from the surface of the patient's skin. However, if device 100 is implanted deeper than 2 cm from the surface of the patient's skin, micro surgery may be used to remove device 100.

In accordance with another embodiment of the invention the external transceiver 200 may be embedded in a mobile device, such as a mobile smartphone device. The mobile device may comprise a dedicated software application installed thereon for the purpose of enabling data communication between the external transceiver 200 and the device 100. The mobile device may further comprise the necessary user interface for activating the device 100 to generate and transmit an electrical signal to stimulate a nerve tissue in a patient.

It is to be understood that the above embodiments have been provided only by way of exemplification of this invention, such as those detailed below, and that further modifications and improvements thereto, as would be apparent to persons skilled in the relevant art, are deemed to fall within the broad scope and ambit of the present invention described. In particular, the following additions and/or modifications can be made without departing from the scope of the invention:
The electrodes may be suitably shaped and need not be needle-shaped—for example, the electrodes may be rod-shaped or elliptically-shaped.
The housing can be globular or a polyhedron, and need not be limited to having an elliptical cross-section as shown in the figures.
The electrical components in the device may be electronically arranged in series or parallel.
The electrodes may be arranged in any particular manner on the housing so long as the therapeutic effect of the device is achieved.

Furthermore, although individual embodiments have been discussed it is to be understood that the invention covers combinations of the embodiments that have been discussed as well.

The invention described herein may include one or more range of values (e.g. temperature). A range of values will be understood to include all values within the range, including the values defining the range, and values adjacent to the range which lead to the same or substantially the same outcome as the values immediately adjacent to that value which defines the boundary to the range.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

1. A nerve stimulation system for treating pain in a patient, the system comprising an implantable device having:
a housing;
a power source;
a processor configured to control the impedance in the device and to optimize ground loop impedance; and
at least one stimulation electrode arranged on the housing and in electrical communication with the power source and the processor, the stimulation electrode adapted to transmit an electrical signal for stimulating at least one nerve cell of the patient,
wherein the power in the power source is wirelessly generated.

2. The nerve stimulation system of claim 1, wherein the device further comprises at least one reference electrode arranged on the housing, wherein the housing has a first end and a second end, and wherein the stimulation electrode is arranged at the first end and the reference electrode is arranged at the second end.

3. (canceled)

4. The nerve stimulation system according to claim 1, the system further comprising at least one transceiver, wherein the device is configured to be in data communication with the transceiver, and the transceiver configured to provide instructions to the device to generate and transmit the electrical signal for stimulating at least one nerve cell in the patient.

5. The nerve stimulation system of claim 4, wherein the transceiver is configured to induce electrical power in the device.

6. (canceled)

7. The nerve stimulation system of claim 4, wherein the processor is configured to wirelessly communicate with the transceiver via radio frequency (RF).

8. The nerve stimulation system of claim 4, wherein the processor is configured to generate the electrical signal.

9. The nerve stimulation system of claim 1, wherein the device includes a temperature transducer configured to monitor the temperature of the device, or optionally the temperature of the environment surrounding the device.

10. (canceled)

11. The nerve stimulation system of claim 1, the device having more than one stimulation electrode.

12. The nerve stimulation system of claim 11, wherein each of the stimulation and reference electrodes has a diameter ranging from 1 μm to 200 μm in one dimension and wherein the distance between each stimulation electrode ranges between 1 μm to 500 μm.

13. The nerve stimulation system of claim 11, wherein the device is configured to select the stimulation electrodes for transmission of the electrical signal.

14. (canceled)

15. The nerve stimulation system of claim 13, wherein the transceiver is configured to provide instructions to the device for the selection of the stimulation electrodes for transmission of the electrical signal.

16. The nerve stimulation system of claim 4, wherein the transceiver is configured to receive data from the device.

17. A nerve stimulation implantable device comprising:
a housing;
a power source;
a processor configured to control the impedance in the device and to optimize ground loop impedance; and
at least one stimulation electrode arranged on the housing and in electrical communication with the power source and the processor, the stimulation electrode adapted to transmit an electrical signal for stimulating at least one nerve cell of the patient,
wherein the power in the power source is wirelessly generated.

18. The nerve stimulation implantable device of claim 17, wherein the device further comprises at least one reference electrode arranged on the housing, wherein the housing has a first end and a second end, and wherein the stimulation electrode is arranged at the first end and the reference electrode is arranged at the second end.

19. (canceled)

20. The nerve stimulation implantable device according to claim 17, wherein the processor is configured to generate the electrical signal.

21. The nerve stimulation implantable device according to claim 17, wherein the device includes a temperature transducer configured to monitor the temperature of the device, or optionally the temperature of the environment surrounding the device.

22. The nerve stimulation implantable device of claim 17, the device having more than one stimulation electrode.

23. The nerve stimulation implantable device of claim 22, wherein each of the stimulation and reference electrodes has a diameter ranging from 1 μm to 200 μm in one dimension and wherein the distance between each stimulation electrode ranges between 1 μm to 500 μm.

24. The nerve stimulation implantable device of claim 17, wherein the device is configured to select the stimulation electrodes for transmission of the electrical signal.

25. (canceled)

26. A method of treating pain in a patient, the method comprising the steps of:
Implanting at least one implantable device at an implantation site of the patient, the device having:
a housing;
a power source;
a processor configured to control the impedance in the device and to optimize ground loop impedance; and
at least one stimulation electrode arranged on the housing and in electrical communication with the power source and the processor, the stimulation electrode adapted to transmit an electrical signal for stimulating at least one nerve cell of the patient;
wherein the power in the power source is wirelessly generated;
Providing a transceiver, wherein the at least one implantable device is configured to be in data communication with the transceiver; and
Instructing the device via the transceiver to generate and transmit via the at least one stimulation electrode, an electrical signal for stimulating at least one nerve cell in the patient.
